(19) United States
(12) Patent Application Publication
(10) Pub. No.: US 2010/0227925 A1
(43) Pub. Date: Sep. 9, 2010

(54) SENSITIZING COMPOUND AND METHOD FOR INCREASING SEXUAL SENSITIVITY

(76) Inventor: Ruth Chatfield, Fort Lauderdale, FL (US)

Correspondence Address: MAYBACK & HOFFMAN, P.A., 5722 S. FLAMINGO ROAD #232, FORT LAUDERDALE, FL, 33330, US

(21) Appl. No.: 12/429,041

(22) Filed: Apr. 23, 2009

Related U.S. Application Data
(60) Provisional application No. 61/158,534, filed on Mar. 9, 2009.

(51) Int. Cl. A61K 31/198; A61P 15/00

(52) U.S. Cl. 514/563

(57) ABSTRACT

A clitoral sensitizing agent includes a base, L-citrulline, and a promotional vehicle.

### FIELD OF THE INVENTION

This invention relates to compounds and methods for the stimulation of female body parts and, more particularly, to a topically-applied compound available in user-instantly-selectable formula strengths.

### BACKGROUND OF THE INVENTION

Sexual stimulation is a natural and important part of life for humans. It comforts and calms, it brings or keeps couples close together emotionally, as well as physically, and science shows that it is good for one's overall health. For females, sexual stimulation is usually centered around the clitoris, which is a sexual organ located near the anterior junction of the labia minora, above the opening of the urethra and vagina in females. Unlike the penis, which is homologous to the clitoris, the clitoris does not contain the distal portion of the urethra, and functions solely to induce sexual pleasure. However, for a variety of reasons, in some cases, natural stimulation is found to be insufficient or can be enhanced simply for the purpose of increasing pleasure for the woman.

Some pharmaceutical methods of stimulating the clitoris have been attempted but have fallen short of being effective for all users. One reason is an improperly effective compound being used and a second is that the necessary dosage of stimulating substances varies from subject to subject.

Therefore, a need exists to overcome the problems with the prior art as discussed above.

### SUMMARY OF THE INVENTION

Although the invention is illustrated and described herein as embodied in a sensitizing compound and a method for increasing sexual sensitivity, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures. The figures of the drawings are not drawn to scale.

The invention provides a sensitizing compound and method for increasing sexual sensitivity that overcomes the hereinafore-mentioned disadvantages of the heretofore-known devices and methods of this general type.

With the foregoing and other objects in view, there is provided, in accordance with the invention, a clitoral sensitizing agent comprising a base, L-citrulline and a promotional vehicle.

In accordance with another feature of the invention, the base is comprised of one or more compounds selected from a group consisting of propylene glycol, polyethylene glycol, cetyl hydroxyethylcellulose, paraben, cocoa butter, and shea butter.

In accordance with a further feature of the invention, the promotional vehicle is selected from a group consisting of menthol, peppermint oil, spearmint oil, rosemary oil, oregano oil, capsaicin, cinnamon oil, nicotinate esters and niacin.

In accordance with an added feature of the invention, the base is solid at ambient temperature and liquid at human body temperature and the promotional vehicle is menthol.

In accordance with yet a further feature of the invention, the L-citrulline comprises between 1% and 10% of the agent.

In accordance with yet an additional feature of the invention, the promotional vehicle is menthol, wherein the menthol comprises between 0.1% and 10% of the agent, and wherein the L-citrulline comprises between 1% and 10% of the agent.

With the objects of the invention in view, there is also provided a base, a rubefacient, L-citrulline and a promotional vehicle.

In accordance with still another feature of the invention, the rubefacient is selected from one or more of a group consisting of methyl nicontinate, niacin, capsaicin, peppermint oil, cinnamon oil, rosemary oil, and oregano oil.

In accordance with still an added feature of the invention, the base is comprised of propylene glycol and cetyl hydroxyethylcellulose, the promotional vehicle is comprised of menthol, and the rubefacient is selected from a group consisting of nicontinate esters, niacin, capsaicin, peppermint oil, cinnamon oil, rosemary oil, and oregano oil.

With the objects of the invention in view, there is further provided a method of increasing sexual sensitivity that includes providing a sensitizing cream comprising L-citrulline and topically applying the sensitizing cream to a clitoris such that the L-citrulline is absorbed transdermally.

In accordance with an additional feature of the invention, the sensitizing cream is solid at ambient temperature and liquid at human body temperature.

In accordance with another mode, the invention includes providing a promoting cream that includes a promotional vehicle wherein the promoting cream is applied to the clitoris simultaneously with the applying the sensitizing cream at a rate variable to a rate at which the sensitizing cream is applied.

Before the present invention is disclosed and described, it is to be understood that the terminology used herein is for the purpose of describing particular embodiments only and is not intended to be limiting. The terms “a” or “an”, as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

### BRIEF DESCRIPTION OF THE DRAWINGS

The accompanying figures, where like reference numerals refer to identical or functionally similar elements throughout the separate views and which, together with the detailed description below, are incorporated in and form part of the specification, serve to further illustrate various embodiments and to explain various principles and advantages all in accordance with the present invention.

FIG. 1 is a diagram illustrating an L-Citrulline-to-Nitric Oxide cycle.

FIG. 2 is a diagram illustrating estrogen and progesterone levels of the female sex hormone cycle.

FIG. 3 is a block diagram illustrating an exemplary dispenser for delivering user-selectable proportions of components of the presently inventive compound in accordance with an embodiment of the present invention.

### DETAILED DESCRIPTION

Herein various embodiments of the present invention are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

The present invention provides a novel compound of L-Citrulline and menthol and/or other ingredients, such as propylene glycol, as the promotional vehicle, which, together, can be used as an effective clitoral sensitizing agent. More specifically, alternate preparations of menthol and related cooling compounds, with the focus on the absorption of L-Citrulline for Nitric Oxide (NO) production to dilate the blood vessels in the clitoral area, are utilized herein to cause increased clitoral sensitization. Menthol can serve as both a promotional vehicle and an active ingredient.

The present invention, according to an embodiment, advantageously utilizes precursors to L-Arginine in place of L-Arginine. Such precursors can include, but are not limited to L-Citrulline, which is a precursor to L-Arginine via the L-Citrulline-L-Arginine recycle pathway. This pathway also represents a renewable source of L-Arginine in vivo and will produce a better local clitoral vasodilatation. The L-Citrulline-L-Arginine recycle pathway further represents a renewable source of NO. In the vasculature, NO relaxes smooth muscles causing vasodilatation. NO also plays an important role in cell signaling in the nervous system.

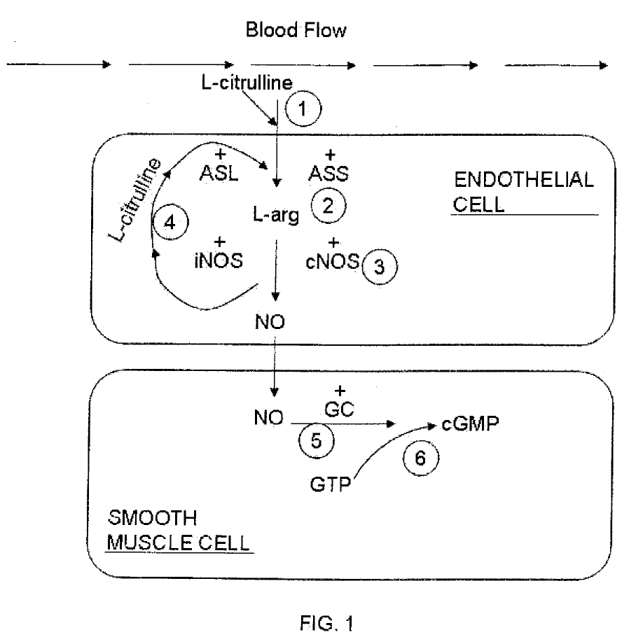

Much has been studied about the L-Arginine-NO pathway, but very little has been studied regarding the complete L-Citrulline-NO cycle. In this cycle, illustrated in the diagram of FIG. 1, L-Citrulline is a byproduct of NO production and, by the actions of two enzymes, argininosuccinate synthase and argininosuccinate lyase, is regenerated into L-Arginine for a renewable source of NO formation via nitric oxide synthase. This is not an exogenous source of L-Arginine, but rather, a renewable way to enter and activate the cycles in vivo.

Specifically, FIG. 1 shows an illustrative representation of the cycles as a whole. In a first stage, L-Citrulline is absorbed through the skin into the circulation. Natural blood flow causes it to permeate into the endothelial cell. In a second stage, L-Citrulline+argininosuccinate synthase (ASS) and argininosuccinate lysase (ASL) regenerates into L-Arginine (L-Arg). Next, in a third stage, L-Arg+2 forms of Nitric Oxide Synthase, iNOS and eNOS, form Nitric Oxide (NO). L-Citrulline is formed in a fourth stage as a byproduct, the byproduct being a renewable L-Citrulline moving back to the first stage. In a fifth stage, NO permeates into the smooth muscle and binds with and activates guanyl cyclase (GC). Finally, in a sixth stage, GC catalyzes the dephosphorylation of GTP to form cGMP, which causes smooth muscle relaxation and vasodilation. Locally, this increases clitoral sensitization.

The Citrulline-NO cycle functions in many different types of cells. It has previously been demonstrated in endothelial cells. Argininosuccinate synthase (ASS) catalyzes the condensation of Citrulline and aspartate to form argininosuccinate, the immediate precursor of arginine, at the expense of an ATP molecule. Initially discovered in the liver as the limiting step of the urea cycle, it is now known to exist in almost all organisms. In microorganisms it is primarily used in the synthesis of arginine.

Potentially, oils, such as peppermint or cinnamon, for instance, could cause the menthol-like effect on the mucous membrane to facilitate or promote the topical absorption of L-Citrulline. In addition, known minor skin irritants can cause a profound reaction when topically applied to mucous membrane, such as redness, irritation, and reflex vasodilatation. This irritant reaction associated with vasodilatation shares some similarities with the menthol effect, and could quite effectively substitute for the menthol in promoting L-Citrulline absorption and actions.

The invention may further include alternate preparations for the base of the vehicle. For instance, cocoa butter has a melting point at body temp and is thick at room temperature and could, in one embodiment, serve as a base of the vehicle. Such biologically-active agents (menthol or its substitutes) and L-Citrulline can be compounded in a non-biologically-active base, or in a biologically active base that promotes absorption, i.e., a vehicle. Any base or vehicle is intended to liquefy at body heat and in the presence of moisture present in mucous membrane when topically applied to mucous-membrane tissue. An active vehicle may be engineered that synergistically functions to promote the absorption and actions of menthol, menthol-related compounds, minor skin irritants, L-Citrulline, or any combination of these.

There are solid/liquid state dynamics for the topical delivery of clitoral sensitizing compounds which cover any of the potential compounds for topical application to sensitize the clitoris because they can have different solid/liquid states at ambient and at body temperatures. Liquefied compounds are readily absorbed, dependent on various other factors described. A gel/cream or liquid compound could be directly applied to the clitoris for topical absorption. The gel/cream must liquefy before absorption can begin to occur. The dynamics of how rapidly a compound transforms from a gel/cream state to a liquid state could be controlled to gain an almost immediate effect.

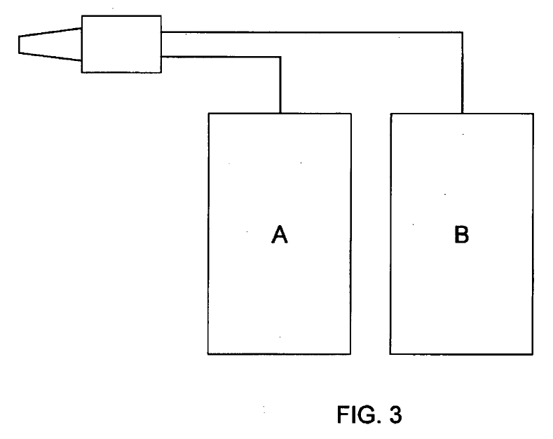

Because the clitoral-sensitizing compounds are also intended to function independently without the preferred intercourse-related physical stimulation of the clitoris, in accordance with an embodiment of the present invention, the menthol/L-Citrulline compounds may also be provided in various strengths, therefore addressing the needs/desires of most users. It is therefore envisioned that the presently-inventive compound be provided in a dispenser, as representatively shown in FIG. 3, that allows a user to variably select the proportion of two (A & B) or more chambers containing ingredients that are combined to form the inventive compounds. One example of a suitable dispenser is described in U.S. Pat. Nos. 7,222,752 and 7,461,987. In these dispenser devices, reservoirs contain respective substances, pumps are associated with the respective reservoirs, and a pushbutton engages the pumps. A dispenser head rotates between settings to position an interior disc at a premeasured angle. The angle determines the extent to which each pump will be engaged when the pushbutton is pressed, thereby, precisely affecting the amount of each substance that is evacuated from its respective reservoir. Mixing ratios from 0 to 100% for each substance, in as little as 5% increments can be preset. By properly setting the dispenser head. Of course, other devices may also be used to achieve the same or similar ratio-setting controls.

When compound ratio settings are available, the menthol may be compounded in multiple strengths, ranging, for instance, from about 0.1% to about ¹⁰⁰%. The L-Citrulline may also be compounded in multiple strengths ranging from, for instance, about 1% to about 10%. The niacin, or any other suitable rubefacient (vasodilator, thus increasing absorption rate and providing a warming effect), PEG8, which becomes a warming agent when mixed with water, or methyl nicotinate may also be in ranges to speed the absorption as well as nullify the cooling effect of the menthol. By combining the inventive formulation with a variable delivery system, the user is advantageously able to specifically quantify the amount of sensitization required for their pleasure.

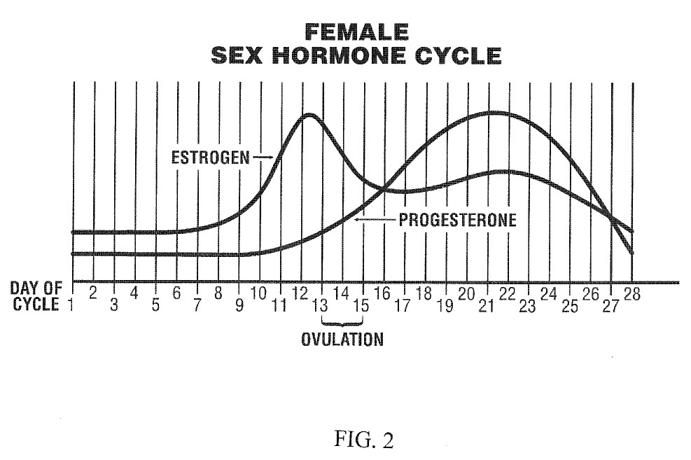

One example of when varying the desired stimulation amount of pharmaceutically-effective substances of the present invention is during or near a woman's monthly cycle. As shown in FIG. 2, when progesterone hormone levels are higher just before a woman's cycle (highest at day 22), less sensitization is required. Just after the beginning of the monthly cycle, more sensitization is needed because of the precipitous drop in hormones levels. The present invention advantageously allows for immediate user variation of the compound being dispensed.

The apparatus and methods to deliver a topical preparation to the clitoris may include of a thickened liquid compound that may be directly applied to the undercarriage of the clitoris regardless of the use of the multiple-use delivery system.

According to embodiments of the present invention, alternate preparations of menthol and related cooling compounds include a class having a single-source of botanical or essential oils that can be used individually or as a combination of several oils such as: Peppermint oil and/or cinnamon oil as compounds. Any of these referenced oils could cause the menthol-like effect on the mucous membrane to facilitate or promote the topical absorption of L-Citrulline. The addition of a second vasodilating agent, topical niacin in menthol or methyl nicotinate, adds a secondary effect to the formulations. If methyl nicotinate or niacin are added, it also increases the absorption of the L-Citrulline and increases the rate of absorption. A second beneficial effect is that this secondary increase in vasodilatation also aids to decrease the initial effect of cooling by menthol and provides a warming sensation that some women find more pleasurable. It also prolongs the effects of the initial vasodilatation from L-Citrulline and thus increases the time of sensitization.

Therefore, according to an embodiment, the present invention includes the step of applying to the clitoris a topical clitoral sensitizing combination including essentially L-Citrulline and a cooling agent that includes menthol. The L-Citrulline, in one example, is less than about 10% of the combination and the cooling agent can include peppermint oil, peppermint flavor, or cinnamon oil, for instance. In one embodiment, the topical clitoral sensitizing compound includes less than about 10% menthol and less than about 10% L-Citrulline. Other applications provide a compound with less than about 5% menthol and less than about 5% L-Citrulline.

In yet another embodiment, the topical clitoral sensitizing combination according to the present invention, includes less than 5% menthol, less than 5% L-Citrulline, and less than 5% topically-applied niacin or other derivatives such as methyl nicotinate.

In accordance with yet another embodiment of the present invention, the inventive compound includes less than about 5% menthol, less than about 5% L-Citrulline, and less than about 10% PEGS.

It is envisioned that the delivery devices provided in accordance with the present invention would have a cap that would seal the device before the initial use, and protect the applicator tip from contamination before use. In addition, multi-use devices would have a resealing cap.

### EXAMPLE 1

| INGREDIENTS: | % |
|---|---|
| Propylene Glycol | 92.22 |
| Cetyl Hydroxyethylcellulose | 4.00 |
| Purified Water | 3.00 |
| Menthol | 0.50 |
| Peppermint Flavor 3502 | 0.12 |
| Niacin | 0.10 |
| Methyl Paraben | 0.05 |
| Tromethamine | 0.01 |

### EXAMPLE 2

| INGREDIENTS: | % |
|---|---|
| Propylene Glycol | 92.77 |
| Cetyl Hydroxyethylcellulose | 4.00 |
| Purified Water | 3.00 |
| Menthol | 0.10 |
| Niacin | 0.05 |
| Methyl Paraben | 0.05 |
| Peppermint Flavor 3502 | 0.03 |

The present invention comprises a compound using apparatus such as a dual chambered proportional mixing apparatus to give any number of different formulas and combinations in one dual chamber container. The apparatus allows the consumer to deliver any number of combinations from chamber A and chamber B from a dial system allowing between 0% to 100% from chamber A and inversely 0% to 100% from chamber B. Each chamber will have a different formulation using similar ingredients (one very light and one very intense) allowing the user to provide numerous combinations and strengths.

What is claimed is:

1. A clitoral sensitizing agent comprising:
a base, L-citrulline and a promotional vehicle.

2. The clitoral sensitizing agent of claim 1, wherein the base is comprised of one or more compounds selected from a group consisting of propylene glycol, polyethylene glycol, cetyl hydroxyethylcellulose, paraben, cocoa butter, and shea butter.

3. The clitoral sensitizing agent of claim 1, wherein the promotional vehicle is selected from a group consisting of menthol, peppermint oil, spearmint oil, rosemary oil, oregano oil, capsaicin, cinnamon oil, nicotinate esters and niacin.

4. The clitoral sensitizing agent of claim 1, wherein the base is solid at ambient temperature and liquid at human body temperature and the promotional vehicle is menthol.

5. The clitoral sensitizing agent of claim 1, wherein the L-citrulline comprises between 1% and 10% of the agent.

6. The clitoral sensitizing agent of claim 1, wherein the promotional vehicle is menthol, wherein the menthol comprises between 0.1% and 10% of the agent, and wherein the L-citrulline comprises between 1% and 10% of the agent.

7. A clitoral sensitizing agent comprising:
a base, a rubefacient, L-citrulline and a promotional vehicle.

8. The clitoral sensitizing agent of claim 7, wherein the base is comprised of one or more compounds selected from a group consisting of propylene glycol, polyethylene glycol, cetyl hydroxyethylcellulose, paraben, cocoa butter, and shea butter.

9. The clitoral sensitizing agent of claim 7, wherein the promotional vehicle is selected from a group consisting of menthol, peppermint oil, spearmint oil, rosemary oil, oregano oil, capsaicin, cinnamon oil, nicotinate esters and niacin.

10. The clitoral sensitizing agent of claim 7, wherein the rubefacient is selected from one or more of a group consisting of methyl nicontinate, niacin, capsaicin, peppermint oil, cinnamon oil, rosemary oil, and oregano oil.

11. The clitoral sensitizing agent of claim 7, wherein the base is solid at ambient temperature and liquid at human body temperature and the promotional vehicle is menthol.

12. The clitoral sensitizing agent of claim 7, wherein the L-citrulline comprises between 1% and 10% of the agent.

13. The clitoral sensitizing agent of claim 7, wherein the promotional vehicle is menthol, wherein the menthol comprises between 0.1% and 10% of the agent, and wherein the L-citrulline comprises between 1% and 10%. of the agent.

14. The clitoral sensitizing agent of claim 13, wherein the rubefacient is niacin.

15. The clitoral sensitizing agent of claim 14, wherein the agent is solid at ambient temperature and liquid at human body temperature.

16. The clitoral sensitizing agent of claim 7, wherein the base is comprised of propylene glycol and cetyl hydroxyethylcellulose, the promotional vehicle is comprised of menthol, and the rubefacient is selected from a group consisting of nicontinate esters, niacin, capsaicin, peppermint oil, cinnamon oil, rosemary oil, and oregano oil.

17. A method of increasing sexual sensitivity comprising:
a. providing a sensitizing cream comprising L-citrulline; and
b. topically applying the sensitizing cream to a clitoris such that the L-citrulline is absorbed transdermally.

18. The method of claim 17, wherein the sensitizing cream is solid at ambient temperature and liquid at human body temperature.

19. The method of claim 17, further comprising:
providing a promoting cream comprising a promotional vehicle wherein the promoting cream is applied to the clitoris simultaneously with the applying the sensitizing cream at a rate variable to a rate at which the sensitizing cream is applied.

20. The method of claim 19, wherein the promoting cream comprises menthol and a rubefacient.
